(19) United States
(12) Patent Application Publication
(10) Pub. No.: US 2011/0263699 A1
(43) Pub. Date: Oct. 27, 2011

(54) 1-Phenyl-2-DimethylaminomethylCyclohexane Compounds and Therapies for Depressive Symptoms, Pain and Incontinence

(75) Inventors: Elmar Friderichs, Stolberg (DE); Wolfgang Strassburger, Wuerselen (DE); Ulrich Jahnel, Langerwehe (DE); Derek Saunders, Aachen (DE); Hagen-Heinrich Hennies, Simmerath (DE); Werner Englberger, Stolberg (DE); Helmut Buschmann, Esplugues de Llobregat (ES); Joerg Holenz, Vilanova i la Geltru (ES); Oswald Zimmer, Wuerselen (DE)

(73) Assignee: GRUENENTHAL GmbH, Aachen (DE)

(21) Appl. No.: 13/175,807

(22) Filed: Jul. 1, 2011

Related U.S. Application Data
(63) Continuation of application No. 11/037,038, filed on Jan. 19, 2005, now abandoned.
Application No. 11/037,038 is a continuation of application No. PCT/EP2003/007720, filed on Jul. 16, 2003.

(30) Foreign Application Priority Data
Jul. 19, 2002 (DE) 102 33 048.4

(51) Int. Cl. A61K 31/135; C07C 291/04; C07C 211/17; A61P 13/00; C07C 305/24; A61K 31/255; A61P 25/24; A61P 29/00; A61K 31/351; C07D 309/10

(52) U.S. Cl. 514/459; 514/644; 514/646; 564/299; 549/417; 564/443; 558/37; 514/517

(57) ABSTRACT

Metabolites of [2-(3-methoxyphenyl)-cyclohexylmethyl]dimethylamine as free bases and/or in the form of physiologically acceptable salts, corresponding medicaments, the use of [2-(3-methoxyphenyl)-cyclohexylmethyl]-dimethylamine and the metabolites thereof for producing a medicament used for treating depression, and related methods of treating depression, pain and urinary incontinence.

### CROSS REFERENCE TO RELATED APPLICATIONS

This application is a continuation of application Ser. No. 11/037,038, filed Jan. 19, 2005, which was a continuation of International Patent Application No. PCT/EP2003/007720, filed Jul. 16, 2003, designating the United States of America, and published in German on Jan. 29, 2004 as WO 2004/009067 A1, the entire disclosure of which is incorporated herein by reference. Priority is claimed based on Federal Republic of Germany Patent Application No. 102 33 048.4, filed Jul. 19, 2002.

### FIELD OF THE INVENTION

The invention relates to metabolites of [2-(3-methoxyphenyl)-cyclohexylmethyl]-dimethylamine as free bases and/or in the form of physiologically acceptable salts, corresponding medicaments and the use of [2-(3-methoxyphenyl)-cyclohexylmethyl]-dimethylamine and its metabolites for the preparation of a medicament for treatment of depressions and methods for treatment of depressions.

### BACKGROUND OF THE INVENTION

Depression is an affectivity disorder with which a depressive syndrome occupies the foreground, depressive meaning associated with depression or of sad mood. The antidepressants used for therapy are also important adjuvants for pain therapy (R. van Schayck et al. 1998, MMP, 10, 304-313; Jung et al. 1997, J. Gen. Intern. Med. 12, 384-389; Onghena and Van Houdenhove 1992, Pain, 49, 205-219; Feuerstein 1997, Diagnostik and Therapie, 3, 213-225; Rowbotham 1997, The Pain Medicine Journal Club Journal, 3, 119-122), in particular in cases of chronic states of pain, since the lasting pain load can lead to a depressive mood in the patient. This is particularly often the case with cancer pain patients (Berard 1996, Int. Med. J., 3, 257-259). Since there has hitherto been no painkiller with a clinically relevant antidepressant action component, antidepressants must be added to the administration of analgesics as additional medication. Since chronic pain patients often require a large number of various medicaments, the additional administration of the antidepressant leads to a further stress on the organism. For this reason and to improve compliance, an antidepressant action component already present in an analgesic substance would be of particular advantage.

The basis of the antidepressant action is the inhibition of the reuptake of noradrenaline and serotonin. Opioids having a moderate inhibition of the uptake, such as e.g. tramadol, have been known for a relatively long time and are employed therapeutically as potent analgesics having a low addiction and dependency potential (Raffa et al. 1992, Journal of Pharmacology and experimental Therapeutics, 260, 278-285; Raffa and Friderichs 1996, Pain Review, 3, 249-271). However, the uptake-inhibiting component is not potent enough to be able to initiate a clinically relevant antidepressant effect.

In addition to the actual antidepressant action, inhibition of the reuptake of noradrenaline and serotonin also leads to an independent analgesic action, in that descending pain inhibition pathways are activated at the level of the spinal marrow. In combination with opiates, which also inhibit transmission of pain at the spinal marrow level, this leads to a mutual potentiation of the pain inhibition mechanisms and therefore to a particularly effective analgesia.

An opioid substance having a relatively potent NA and/or 5HT uptake-inhibiting component which displays an antidepressant activity (e.g. demonstrated in a suitable behaviour model in animal studies) would therefore be particularly favourable in this indication, in particular treatment of depressions, precisely also in combination with pain therapy.

### SUMMARY OF THE INVENTION

One object of the present invention is to provide substances, in particular opiod substances, which are suitable for treatment of depression, in particular those which combine this activity with analgesic activity.

Surprisingly, it has now been found that 2-(3-methoxyphenyl)-cyclohexylmethyl)-dimethylamine and also its metabolites, and in this context in particular 3-(2-dimethylaminomethyl-cyclohexyl)-phenol, have a therapeutically relevant antidepressant action component.

The invention accordingly provides the use of
3-(2-dimethylaminomethyl-cyclohexyl)-phenol,
(1R,2R)-3-(2-dimethylaminomethyl-cyclohexyl)-phenol,
[2-(3-methoxyphenyl)-cyclohexylmethyl]-dimethylamine,
(1R,2R)-[2-(3-methoxyphenyl)-cyclohexylmethyl]-dimethylamine,
sulfuric acid mono-[3-(2-dimethylaminomethyl-cyclohexyl)-phenyl]ester,
sulfuric acid mono-(1R,2R)-[3-(2-dimethylaminomethyl-cyclohexyl)-phenyl]ester,
3-(2-methylaminomethyl-cyclohexyl)-phenol,
(1R,2R)-3-(2-methylaminomethyl-cyclohexyl)-phenol,
3-(2-dimethylaminomethyl-cyclohexyl)-phenol N-oxide,
(1R,2R)-3-(2-dimethylaminomethyl-cyclohexyl)-phenol N-oxide,
6-[3-(2-dimethylaminomethyl-cyclohexyl)-phenoxy]-3,4,5-trihydroxy-tetrahydropyran-2-carboxylic acid,
6-[(1R,2R)-3-(2-dimethylaminomethyl-cyclohexyl)-phenoxy]-3,4,5-trihydroxy-tetrahydropyran-2-carboxylic acid,
4-(2-dimethylaminomethyl-cyclohexyl)-catechol,
(1R,2R)-4-(2-dimethylaminomethyl-cyclohexyl)-catechol,
3-(2-aminomethyl-cyclohexyl)-phenol,
(1R,2R)-3-(2-aminomethyl-cyclohexyl)-phenol,
4-(2-dimethylaminomethyl-cyclohexyl)-benzene-1,2-diol,
(1R,2R)-4-(2-dimethylaminomethyl-cyclohexyl)-benzene-1,2-diol,
C-[2-(3-methoxy-phenyl)-cyclohexyl]-methylamine,
(1R,2R)—C-[2-(3-methoxy-phenyl)-cyclohexyl]-methylamine,
[2-(3-methoxy-phenyl)-cyclohexylmethyl]-methyl-amine,
(1R,2R)-[2-(3-methoxy-phenyl)-cyclohexylmethyl]-methyl-amine,
[2-(3-methoxy-phenyl)-cyclohexylmethyl]-dimethyl-amine; N-oxide or
(1R,2R)-[2-(3-methoxy-phenyl)-cyclohexylmethyl]-dimethyl-amine; N-oxide;
   optionally in the form of their racemates, their pure stereoisomers, in particular enantiomers or diastereomers, or in the form of mixtures of the stereoisomers, in particular the enantiomers or diastereomers, in any desired mixture ratio;
   in the form shown or in the form of their acids or their bases or in the form of their salts, in particular the physiologically acceptable salts, or in the form of their solvates, in particular the hydrates;
   for the preparation of a medicament for treatment of depression and methods of treatment involving administration of the same.

In this context it is particularly preferable if the compounds used are in the form of R,R, preferably 1R,2R stereoisomers.

The term salt is to be understood as meaning any form of the active compound according to the invention in which this assumes an ionic form or is charged and coupled with a counter-ion (a cation or anion) or is in solution. This is also to be understood as meaning complexes of the active compound with other molecules and ions, in particular complexes which are complexed via ionic interactions. In particular, this is understood as meaning (and this is also a preferred embodiment of this invention) physiologically acceptable salts, in particular physiologically acceptable salts with cations or bases and physiologically acceptable salts with anions or acids or also a salt formed with a physiologically acceptable acid or a physiologically acceptable cation. The preferred salt of the compounds used is the hydrochloride.

Physiologically acceptable is to be understood as meaning that the substance, in particular the salt as such, is acceptable when used in humans or mammals, that is to say, for example, does not have a non-physiological (e.g. toxic) action.

In the context of this invention, the term of physiologically acceptable salt with anions or acids is understood as meaning at least one of the compounds according to the invention—usually protonated, for example on the nitrogen—as the cation with at least one anion, which are physiologically acceptable—especially when used in humans and/or mammals. In particular, in the context of this invention by this there is understood the salt formed with a physiologically acceptable acid, namely salts of the particular active compound with inorganic or organic acids which are physiologically acceptable—especially when used in humans and/or mammals. Examples of physiologically acceptable salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid, citric acid, glutamic acid, 1,1-dioxo-1,2-dihydro1b6-benzo[d]isothiazol-3-one (saccharic acid), monomethylsebacic acid, 5-oxo-proline, hexane-1-sulfonic acid, nicotinic acid, 2-, 3- or 4-aminobenzoic acid, 2,4,6-trimethyl-benzoic acid, a-liponic acid, acetylglycine, acetylsalicylic acid, hippuric acid and/or aspartic acid. The hydrochloride salt is particularly preferred.

In the context of this invention, the term of salt formed with a physiologically acceptable acid is understood as meaning salts of the particular active compound with inorganic or organic acids which are physiologically acceptable—especially when used in humans and/or mammals. The hydrochloride is particularly preferred. Examples of physiologically acceptable acids are: hydrochloric acid, hydrobromic acid, sulfuric acid, methanesulfonic acid, formic acid, acetic acid, oxalic acid, succinic acid, tartaric acid, mandelic acid, fumaric acid, lactic acid, citric acid, glutamic acid, 1,1-dioxo-1,2-dihydro1λ⁶-benzo[d]isothiazol-3-one (saccharic acid), monomethylsebacic acid, 5-oxo-proline, hexane-1-sulfonic acid, nicotinic acid, 2-, 3- or 4-aminobenzoic acid, 2,4,6-trimethyl-benzoic acid, a-liponic acid, acetylglycine, acetylsalicylic acid, hippuric acid and/or aspartic acid.

In the context of this invention, the term of physiologically acceptable salt with cations or bases is understood as meaning salts of at least one of the compounds according to the invention—usually of a (deprotonated) acid—as the anion with at least one preferably inorganic cation which are physiologically acceptable—especially when used in humans and/or mammals. The salts of the alkali metals and alkaline earth metals and also with NH₄⁺ are particularly preferred, but especially (mono-) or (di-)sodium, (mono-) or (di-)potassium, magnesium or calcium salts.

In the context of this invention, the term of salt formed with a physiologically acceptable cation is understood as meaning salts of at least one of the particular compounds as the anion with at least one inorganic cation which is physiologically acceptable—especially when used in humans and/or mammals. The salts of the alkali metals and alkaline earth metals and also NH₄⁺ are particularly preferred, but especially (mono-) or (di-)sodium, (mono-) or (di-)potassium, magnesium or calcium salts.

According to the present investigations, the substances used, and in particular (1R,2R)-3-(2-dimethylaminomethyl-cyclohexyl)-phenol, are potent analgesics and antidepressants, that is to say have an additional and clinically relevant antidepressant action component. In particular, (1R,2R)-3-(2-dimethylaminomethyl-cyclohexyl)-phenol can be employed in cases of moderate to severe acute and chronic pain and renders possible treatment of the depressive concomitant symptoms with chronic states of pain. It therefore leads to a significant improvement in the treatment of chronic pain, since antidepressants hitherto had to be added as additional medicaments to those of pain therapy. (1R,2R)-3-(2-Dimethylaminomethyl-cyclohexyl)-phenol and the other substances used according to the invention can therefore also be used as genuine antidepressants independently of their analgesic action.

The invention also provides metabolites, in particular of [2-(3-methoxyphenyl)-cyclohexylmethyl]-dimethylamine and 3-(2-dimethylaminomethyl-cyclohexyl)-phenol chosen from
sulfuric acid mono-[3-(2-dimethylaminomethyl-cyclohexyl)-phenyl]ester,
sulfuric acid mono-(1R,2R)-[3-(2-dimethylaminomethyl-cyclohexyl)-phenyl]ester,
3-(2-methylaminomethyl-cyclohexyl)-phenol,
(1R,2R)-3-(2-methylaminomethyl-cyclohexyl)-phenol,
3-(2-dimethylaminomethyl-cyclohexyl)-phenol N-oxide,
(1R,2R)-3-(2-dimethylaminomethyl-cyclohexyl)-phenol N-oxide,
6-[3-(2-dimethylaminomethyl-cyclohexyl)-phenoxy]-3,4,5-trihydroxy-tetrahydropyran-2-carboxylic acid,
6-[(1R,2R)-3-(2-dimethylaminomethyl-cyclohexyl)-phenoxy]-3,4,5-trihydroxy-tetrahydropyran-2-carboxylic acid,
4-(2-dimethylaminomethyl-cyclohexyl)-catechol,
(1R,2R)-4-(2-Dimethylaminomethyl-cyclohexyl)-catechol,
3-(2-aminomethyl-cyclohexyl)-phenol,
(1R,2R)-3-(2-aminomethyl-cyclohexyl)-phenol,
4-(2-dimethylaminomethyl-cyclohexyl)-benzene-1,2-diol,
(1R,2R)-4-(2-dimethylaminomethyl-cyclohexyl)-benzene-1,2-diol,
C-[2-(3-methoxy-phenyl)-cyclohexyl]-methylamine,
(1R,2R)—C-[2-(3-methoxy-phenyl)-cyclohexyl]-methylamine,
[2-(3-methoxy-phenyl)-cyclohexylmethyl]-methyl-amine,
(1R,2R)-[2-(3-methoxy-phenyl)-cyclohexylmethyl]-methyl-amine,
[2-(3-methoxy-phenyl)-cyclohexylmethyl]-dimethyl-amine; N-oxide or
(1R,2R)-[2-(3-methoxy-phenyl)-cyclohexylmethyl]-dimethyl-amine N-oxide;
   optionally in the form of their racemates, their pure stereoisomers, in particular enantiomers or diastereomers, or in the form of mixtures of the stereoisomers, in particular the enantiomers or diastereomers, in any desired mixture ratio; in the form shown or in the form of their acids or their bases or in the form of their salts, in particular the physiologically acceptable salts, or in the form of their solvates, in particular the hydrates.

Metabolites according to the invention which are in the form of R,R, preferably 1R,2R stereoisomers are particularly preferred.

The metabolites according to the invention are physiologically acceptable. The invention therefore also provides a medicament comprising as the active compound at least one metabolite according to the invention and optionally additives and auxiliary substances.

In particular, the invention accordingly provides a medicament comprising as the active compound at least one compound chosen from
sulfuric acid mono-[3-(2-dimethylaminomethyl-cyclohexyl)-phenyl]ester,
sulfuric acid mono-(1R,2R)-[3-(2-dimethylaminomethyl-cyclohexyl)-phenyl]ester,
3-(2-methylaminomethyl-cyclohexyl)-phenol,
(1R,2R)-3-(2-methylaminomethyl-cyclohexyl)-phenol,
3-(2-dimethylaminomethyl-cyclohexyl)-phenol N-oxide,
(1R,2R)-3-(2-dimethylaminomethyl-cyclohexyl)-phenol N-oxide,
6-[3-(2-dimethylaminomethyl-cyclohexyl)-phenoxy]-3,4,5-trihydroxy-tetrahydropyran-2-carboxylic acid,
6-[(1R,2R)-3-(2-dimethylaminomethyl-cyclohexyl)-phenoxy]-3,4,5-trihydroxy-tetrahydropyran-2-carboxylic acid,
4-(2-dimethylaminomethyl-cyclohexyl)-catechol,
(1R,2R)-4-(2-Dimethylaminomethyl-cyclohexyl)-catechol,
3-(2-aminomethyl-cyclohexyl)-phenol,
(1R,2R)-3-(2-aminomethyl-cyclohexyl)-phenol,
4-(2-dimethylaminomethyl-cyclohexyl)-benzene-1,2-diol,
(1R,2R)-4-(2-dimethylaminomethyl-cyclohexyl)-benzene-1,2-diol,
C-[2-(3-methoxy-phenyl)-cyclohexyl]-methylamine,
(1R,2R)—C-[2-(3-methoxy-phenyl)-cyclohexyl]-methylamine,
[2-(3-methoxy-phenyl)-cyclohexylmethyl]-methyl-amine,
(1R,2R)-[2-(3-methoxy-phenyl)-cyclohexylmethyl]-methyl-amine,
[2-(3-methoxy-phenyl)-cyclohexylmethyl]-dimethyl-amine; N-oxide or
(1R,2R)-[2-(3-methoxy-phenyl)-cyclohexylmethyl]-dimethyl-amine N-oxide;
   optionally in the form of their racemates, their pure stereoisomers, in particular enantiomers or diastereomers, or in the form of mixtures of the stereoisomers, in particular the enantiomers or diastereomers, in any desired mixture ratio; in the form shown or in the form of their acids or their bases or in the form of their salts, in particular the physiologically acceptable salts, or in the form of their solvates, in particular the hydrates;
   and optionally additives and/or auxiliary substances.

It is particularly preferable if the compounds contained therein are in the form of R,R, preferably 1R,2R stereoisomers.

Suitable additives and/or auxiliary substances in the context of this invention are all the substances known to the expert from the prior art for achieving galenical formulations. The choice of these auxiliary substances and the amounts thereof to be employed depend on whether the medicament is to be administered orally, intravenously, intraperitoneally, intradermally, intramuscularly, intranasally, buccally or locally. Formulations in the form of tablets, chewable tablets, coated tablets, capsules, granules, drops, juices or syrups are suitable for oral administration, and solutions, suspensions, easily reconstitutable dry formulations and sprays are suitable for parenteral, topical and inhalatory administration. Suppositories for use in the rectum are a further possibility. The use in a depot in dissolved form, a carrier film or a patch, optionally with the addition of agents which promote penetration through the skin, are examples of suitable forms for percutaneous administration. Examples of auxiliary substances and additives for the oral administration forms are disintegrating agents, lubricants, binders, fillers, mould release agents, optionally solvents, flavourings, sugars, in particular carrier agents, diluents, dyestuffs, antioxidants etc. For suppositories, inter alia, waxes and fatty acid esters can be used, and for compositions for parental administration carrier substances, preservatives, suspension auxiliaries etc. can be used. The amounts of active compound to be administered to patients vary as a function of the weight of the patient, the mode of administration and the severity of the disease. The compounds used according to the invention can be released in a delayed manner from formulation forms which can be used orally, rectally or percutaneously. Corresponding sustained-release formulations, in particular in the form of a “once daily” preparation which has to be taken only once a day, are particularly preferred for the indication according to the invention.

Medicaments which comprise at least 0.05 to 90.0% of the active compound, in particular low active dosages, in order to avoid side effects or analgesic actions, are preferred. 0.1 to 5,000 mg/kg, in particular 1 to 500 mg/kg, preferably 2 to 250 mg/kg of body weight of a least one compound used according to the invention are conventionally administered. However, the administration of 0.01-5 mg/kg, preferably 0.03 to 2 mg/kg, in particular 0.05 to 1 mg/kg, is also preferred and conventional.

Auxiliary substances can be, for example: water, ethanol, 2-propanol, glycerol, ethylene glycol, propylene glycol, polyethylene glycol, polypropylene glycol, glucose, fructose, lactose, sucrose, dextrose, molasses, starch, modified starch, gelatine, sorbitol, inositol, mannitol, microcrystalline cellulose, methylcellulose, carboxymethylcellulose, cellulose acetate, shellac, cetyl alcohol, polyvinylpyrrolidone, paraffins, waxes, naturally occurring and synthetic gums, gum acacia, alginates, dextran, saturated and unsaturated fatty acids, stearic acid, magnesium stearate, zinc stearate, glyceryl stearate, sodium lauryl sulfate, edible oils, sesame oil, coconut oil, groundnut oil, soya bean oil, lecithin, sodium lactate, polyoxyethylene and -propylene fatty acid esters, sorbitan fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potash, calcium phosphate, dicalcium phosphate, potassium bromide, potassium iodide, talc, kaolin, pectin, crospovidone, agar and bentonite.

The medicaments and pharmaceutical compositions according to the invention are prepared with the aid of means, devices, methods and processes which are well-known in the prior art of pharmaceutical formulation, such as are described, for example, in “Remington's Pharmaceutical Sciences”, ed. A. R. Gennaro, 17th ed., Mack Publishing Company, Easton, Pa. (1985), in particular in part 8, chapter 76 to 93.

Thus e.g. for a solid formulation, such as a tablet, the active compound of the medicament, i.e. a compound of the general structure I or one of its pharmaceutically acceptable salts, can be granulated with a pharmaceutical carrier, e.g. conventional tablet constituents, such as maize starch, lactose, sucrose, sorbitol, talc, magnesium stearate, dicalcium phosphate or pharmaceutically acceptable gums, and pharmaceutical diluents, such as e.g. water, in order to form a solid composition which comprises a compound according to the invention or a pharmaceutically acceptable salt thereof in homogeneous distribution. Homogeneous distribution is understood here as meaning that the active compound is uniformly distributed over the entire composition, so that this can easily be divided into unit dose forms, such as tablets, pills or capsules, having the same activity. The solid composition is then divided into unit does forms. The tablets or pills of the medicament according to the invention or of the compositions according to the invention can also be coated or compounded in another manner in order to provide a dose form with delayed release. Suitable coating compositions are, inter alia, polymeric acids and mixtures of polymeric acids with materials such as e.g. shellac, cetyl alcohol and/or cellulose acetate.

Although the medicaments according to the invention show only few side effects, it may be of advantage—should this be necessary at all—for example to avoid certain forms of dependency, also to use morphine antagonists, in particular naloxone, naltrexone and/or levallorphan, in addition to the compounds used.

Since the compounds according to the invention—as stated above—have an analgesic action, the invention also provides the use of a compound according to the invention or of a metabolit according to the invention for the preparation of a medicament for treatment of pain, in particular acute, visceral, chronic or neuropathic pain or cancer pain.

Since the compounds according to the invention moreover also show an activity in cases of urinary incontinence, the invention also provides the use of a compound according to the invention or of a metabolite according to the invention for the preparation of a medicament for treatment of an increased urge to urinate or urinary incontinence.

The invention furthermore also relates to a method for treatment of depression, in which the compounds used according to the invention are used.

The invention likewise also relates to a method for treatment of pain, in particular acute, visceral, chronic or neuropathic pain or cancer pain, in which the compounds or metabolites according to the invention are used.

The invention likewise also relates to a method for treatment of an increased urge to urinate or urinary incontinence, in which the compounds or metabolites according to the invention are used.

2-(3-Methoxyphenyl)-cyclohexylmethyl)-dimethylamine and (1R,2R)-[2-(3-methoxyphenyl)-cyclohexylmethyl)-dimethylamine] and their preparation are known from DE 195 25 137 A1 Example 8 and U.S. Pat. No. 5,733,936 Example 8, where the absolute stereochemistry of the compound (−6) prepared according to Example 8 is certainly correctly (1R,2R) and not (1R,2S). 3-(2-Dimethylaminomethyl-cyclohexyl)-phenol and (1R,2R)-3-(2-dimethylaminomethyl-cyclohexyl)-phenol and their preparation are also known from DE 195 25 137 A1 Example 10 and U.S. Pat. No. 5,733,936, Example 10, where the absolute stereochemistry of the compound (−7) prepared according to Example 10 is certainly correctly (1R,2R) and not (1R,2S).

The compounds which, and the preparation of which, are not yet known from DE 195 25 137 A1 or U.S. Pat. No. 5,733,936 were prepared in accordance with the examples.

The following examples are intended to explain the invention without the subject matter of the invention being limited thereto.

### EXAMPLES

Generally, the purification and the separation of enantiomers in all the processes mentioned as an example is carried out at the various stages with column chromatography, and predominantly HPLC.

### Example 1

### Preparation of (1R,2R)-[2-(3-methoxyphenyl)-cyclohexylmethyl]-methylamine, hydrochloride

[2-(3-Methoxyphenyl)-cyclohexylmethyl]-methylamine, and (1R,2R)-[2-(3-methoxyphenyl)-cyclohexylmethyl]methylamine, in particular the hydrochloride salt thereof, are prepared as follows:

5.67 g (22.9 mmol) (1R,2R)-[2-(3-methoxyphenyl)-cyclohexylmethyl]-dimethylamine as the free base were heated in 389.64 ml toluene with 3.16 ml (25.2 mmol) phenyl chloroformate for 3 h. After cooling and washing, the organic residue was concentrated and stirred at 110° C. with 192.53 ml ethylene glycol and a total of 45.84 ml 5 N NaOH for a total of 8.5 h, with occasional stirring. (1R,2R)-[2-(3-Methoxyphenyl)-cyclohexylmethyl]-methylamine was formed. This was worked up and precipitated as the hydrochloride with TMCS.

### Example 2

### Preparation of (1R,2R)-3-(2-methylaminomethyl-cyclohexyl)-phenol, hydrochloride

3.55 g (15.2 mmol) of the hydrochloride salt of the (1R,2R)-[2-(3-methoxy-phenyl)-cyclohexylmethyl]-methyl-amines according to Example 1 were stirred under reflux in 4.59 ml (47-48%) aqueous HBr for 7.5 h and the mixture was cooled overnight. (1R,2R)-3-(2-Methylaminomethyl-cyclohexyl)-phenol is formed. This was worked up and precipitated as the hydrochloride with TMCS.

### Example 3

### Preparation of sulfuric acid mono-(1R,2R)-[3-(2-dimethylaminomethyl-cyclohexyl)-phenyl]ester

Sulfuric acid mono-[3-(2-dimethylaminomethyl-cyclohexyl)-phenyl]ester or sulfuric acid mono-(1R,2R)-[3-(2-dimethylaminomethyl-cyclohexyl)-phenyl]ester was prepared as follows:

(1R,2R)-3-(2-Dimethylaminomethyl-cyclohexyl)-phenol; hydrochloride was treated with dicyclohexylcarbodiimide (DCC) in H₂SO₄. Sulfuric acid mono-(1R,2R)-[3-(2-dimethylaminomethyl-cyclohexyl)-phenyl]ester was formed.

### Example 4

### Preparation of 6-[(1R,2R)-3-(2-dimethylaminomethyl-cyclohexyl)-phenoxy]-3,4,5-trihydroxy-tetrahydropyran-2-carboxylic acid

6-[3-(2-Dimethylaminomethyl-cyclohexyl)-phenoxy]-3,4,5-trihydroxy-tetrahydropyran-2-carboxylic acid or 6-[(1R,2R)-3-(2-dimethylaminomethyl-cyclohexyl)-phenoxy]-3,4,5-trihydroxy-tetrahydropyran-2-carboxylic acid was prepared as follows:

(1R,2R)-3-(2-Dimethylaminomethyl-cyclohexyl)-phenol; hydrochloride was treated with 3,4,5-tri-O-acetyl-1-a-bromo-D-glucoronic acid methyl ester with 1. LiOH and 2. HOAc. 6-[(1R,2R)-3-(2-Dimethylaminomethyl-cyclohexyl)-phenoxy]-3,4,5-trihydroxy-tetrahydropyran-2-carboxylic acid was formed. Purification was carried out via a Lobar-Lichoprep RP128 column MeOH:H20 system with subsequent HPLC separation.

### Example 5

### Preparation of [2-(3-methoxy-phenyl)-cyclohexylmethyl]-dimethyl-amine; N-oxide

[2-(3-Methoxy-phenyl)-cyclohexylmethyl]-dimethyl-amine; N-oxide or (1R,2R)-[2-(3-methoxy-phenyl)-cyclohexylmethyl]-dimethyl-amine; N-oxide was prepared as follows:

(1R,2R)-[2-(3-Methoxy-phenyl)-cyclohexylmethyl]-dimethyl-amine; hydrochloride was dissolved, and treated with H₂O₂ at room temperature. (1R,2R)-[2-(3-Methoxy-phenyl)-cyclohexylmethyl]-dimethyl-amine; N-oxide was formed.

### Example 6

### Preparation of (1R,2R)-3-(2-dimethylaminomethyl-cychlohexyl)-phenol N-oxide

3-(2-Dimethylaminomethyl-cychlohexyl)-phenol N-oxide or (1R,2R)-3-(2-dimethylaminomethyl-cychlohexyl)-phenol N-oxide was prepared as follows:

(1R,2R)-3-(2-Dimethylaminomethyl-cychlohexyl)-phenol; hydrochloride was dissolved, and treated with H₂O₂ at room temperature. (1R,2R)-3-(2-Dimethylaminomethyl-cychlohexyl)-phenol N-oxide was formed.

### Example 7

### Preparation of 4-(2-dimethylaminomethyl-cyclohexyl)-catechol or 4-(2-dimethylaminomethyl-cyclohexyl)-benzene-1,2-diol

4-(2-Dimethylaminomethyl-cyclohexyl)-catechol or 4-(2-dimethylaminomethyl-cyclohexyl)-benzene-1,2-diol was prepared in accordance with the following reaction equation:

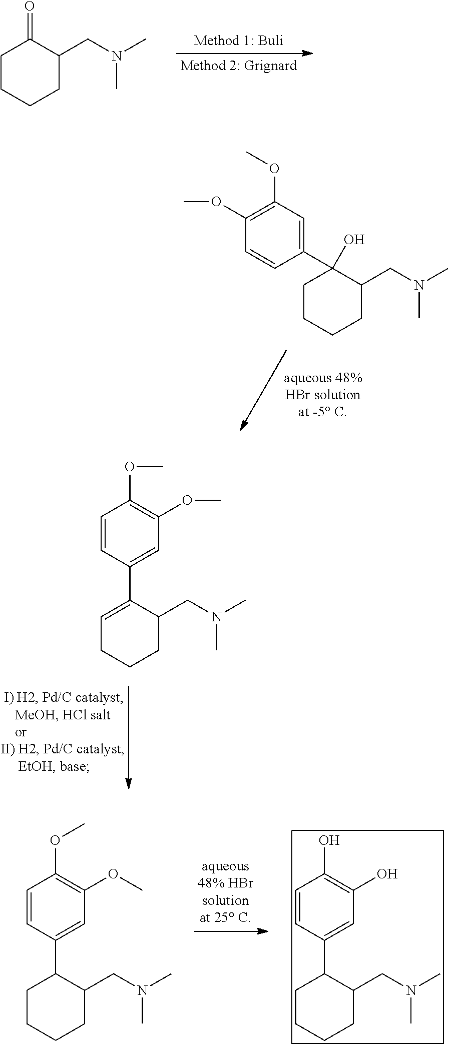

In this, in addition, Method 1 BuLi designates the synthesis via BuLi reagents which is well-known to the expert, and Method 2 Grignard designates the synthesis via Mg reagents which is well-known to the expert.

### Example 8

### Preparation of C-[2-(3-methoxy-phenyl)-cyclohexyl]methylamine

The preparation of C-[2-(3-methoxy-phenyl)-cyclohexyl]-methylamine was carried out in accordance with the following reaction equation:

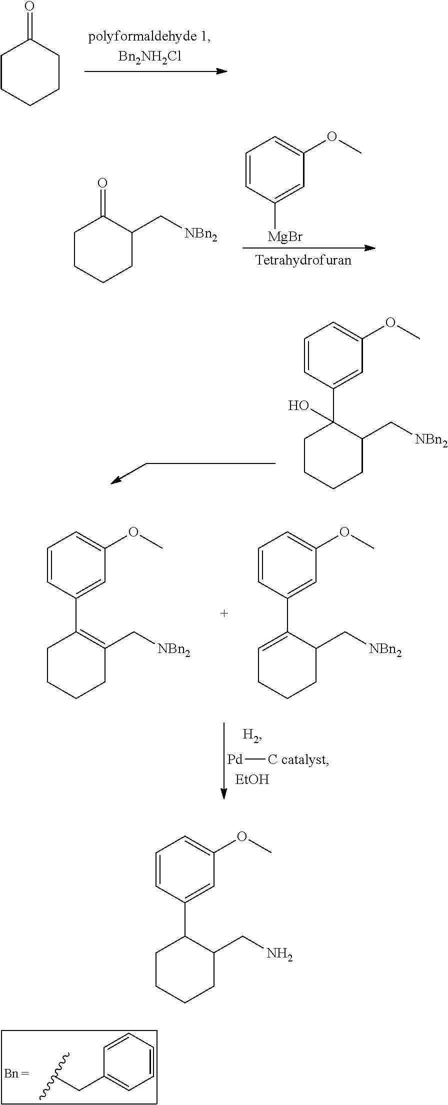

### Example 9

### Preparation of (1R,2R)-3-(2-aminomethyl-cyclohexyl)-phenol

3-(2-Aminomethyl-cyclohexyl)-phenol or (1R,2R)-3-(2-aminomethyl-cyclohexyl)-phenol was prepared in accordance with the following reaction equation:

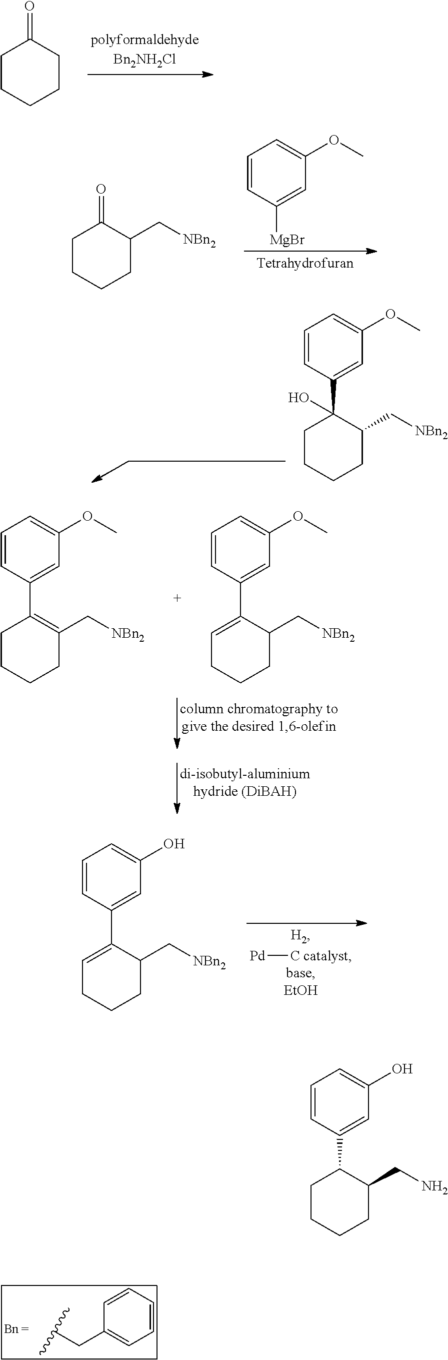

### Example 10

### In Vitro Isolation of the Metabolites

[2-(3-Methoxyphenyl)-cyclohexylmethyl]-dimethylamine; hydrochloride and in another example (1R,2R)-3-(2-dimethylaminomethyl-cyclohexyl)-phenol; hydrochloride was dissolved in TRIS/HCl buffer pH 7.4. MgCl and, where appropriate, the other necessary cofactors, known from the literature, for cytochrome P450 (CytP450) were then added and the batch was incubated with CytP450 3A4 (N-demethylation) and/or CytP450 2D6 (O-demethylation) at 37° C. The batch was then separated via HPLC and the metabolites in the fractions were identified via NMR and then isolated from the fractions.

### Example 11

### In Vivo Isolation of the Metabolites

[2-(3-Methoxyphenyl)-cyclohexylmethyl]-dimethylamine; hydrochloride and in a further example (1R,2R)-3-(2-dimethylaminomethyl-cyclohexyl)phenol; hydrochloride were injected into a mammal. Blood was taken from the mammal and, after the corpuscular constituents had been separated off, this was separated via HPLC and the metabolites in the fractions were identified via NMR and then isolated from the fractions.

### Example 12

### μ-Opioid Receptor Affinity and NA and 5HT Uptake-Inhibiting Components

In addition to the binding to the μ-opioid receptor, which is decisive for an opiod substance, the NA and 5HT uptake-inhibiting components were also investigated for two antidepressants which are used clinically, fluoxetine and desipramine. The results of these standard tests, the procedure of which is adequately known in the literature, are summarized in Table 1:

TABLE 1
Uptake inhibition and opioid receptor binding

| | Kᵢ values (μM) | | | |
|---|---|---|---|---|
| | (1R,2R)-3-(2- | | | |
| | Dimethyl- | (1R,2R)-3-(2- | | |
| | aminomethyl- | Methylamino- | | |
| | cyclohexyl)- | methyl-cyclo- | | |
| | phenol; hydro- | hexyl)-phenol; | | |
| | chloride | hydrochloride | Fluoxetine | Desipramine |
| μ-Opioid | 0.038 | 0.91 | inactive | inactive |
| receptor | (rat) | (human) | | |
| NA uptake | 0.16 | 0.56 | 0.53 | 0.0011 |
| inhibition | | | | |
| 5HT uptake | 0.05 | 9.41 | 0.026 | 1.44 |
| inhibition | | | | |

The 5HT reuptake inhibition precisely of (1R,2R)-3-(2-dimethylaminomethyl-cyclohexyl)-phenol is of the same order of magnitude as the μ-opioid receptor binding of the substance. The substance thus has an exactly balanced ratio of μ-opioid component and uptake inhibition and therefore also has the potential for a particularly effective pain inhibition. The uptake inhibition is of the order of magnitude of antidepressants used clinically and thus leads to an independent antidepressant action component, the substance having a relatively potent inhibiting action on the noradrenaline and, in particular, serotonin reuptake.

### Example 13

### Tail Suspension Test

The tail suspension test detects antidepressant and anxiolytic activity and follows the method of Porsolt et al. (Porsolt R. D., Bertin A., Jalfre M., Behaviourial despair in mice: a primary screening test for antidepressants. Arch. Int. Pharmacodyn. Ther., 229, 327-336, 1977) and Stéru at al. (Stéru L., Chemat R., Thierry B, Simon P. The Tail Suspension Test: a new method for screening antidepressants in mice. Psychopharmacology, 85, 367-370, 1985). Rodents left hanging by the tail rapidly become immobile. Antidepressants reduce the duration of the immobility, while tranquilizers prolong the duration of the immobility. The behaviour of the animals was observed for 6 minutes, preferably with a computerized apparatus (Itemac-TST) developed by Stéru et al. (1985) (see above). Several mice were investigated in parallel and the duration of immobility determined. This parameter is analogous to that in the behavioural despair test (Stéru L., Chemat R., Thierry B., Mico J. A., Lenégre A., Stéru M., Simon P. Porsolt R. D., The automated Tail Suspension Test: a computerized device which differentiates psychotropic drugs. Prog. Neuropsychopharmacol. Exp. Psychiatry, 11, 659-671, 1987). 10-20 mice were investigated per group. Morphine, (1R,2R)-3-(2-dimethylaminomethyl-cyclohexyl)-phenol; hydrochloride and desipramine were administered i.p. 30 minutes before the test and naloxone i.v.

This behavioral model is capable of demonstrating the antidepressant action of the uptake inhibition component in the simultaneous presence of an opioid action. In this behavioural model, antidepressants, as also in other behavioural models, lead to a shortening of the immobility phase, while opioids such as morphine prolong the immobility phase. If both action components are present the two effects overlap, so that only a slight change in motility results. This was eliminated by the opiate component being blocked by pretreatment with naloxone. After the pretreatment with naloxone, the stimulating action of (1R,2R)-3-(2-dimethylaminomethyl-cyclohexyl)-phenol, which was only slight without pretreatment, manifested itself clearly. The substance is therefore very suitable, especially in combination with a pain therapy. The results of these investigations together with reference substances are summarized in Table 2.

TABLE 2
Action of (1R,2R)-3-(2-dimethylaminomethyl-cyclohexyl)-phenol,
imipramine and morphine in the tail suspension test on mice
(10-20 animals/group)

| | Dose | Duration of immobility |
|---|---|---|
| | (mg/kg | % change compared with |
| Substance | i.p.) | the solvent control |
| (1R,2R)-3-(2- | 4.64 | +6% |
| Dimethylaminomethyl- | 10.0 | +12% |
| cyclohexyl)-phenol; | 21.5 | +9% |
| hydrochloride | 31.6 | −13% |
| (1R,2R)-3-(2- | 21.5 | −90% |
| Dimethylaminomethyl- | 31.6 | −85% |
| cyclohexyl)-phenol; | | |
| hydrochloride + 1.0 mg/kg | | |
| i.v. naloxone | | |
| Naloxone 1.0 mg/kg i.v. | — | −15% |
| Morphine | 4.64 | +90% |
| | 10.0 | +145% |
| | 21.5 | +169% |
| Imipramine | 31.6 | −61% |

Table 2 shows that (1R,2R)-3-(2-dimethylaminomethyl-cyclohexyl)-phenol; hydrochloride causes a marked decrease in the duration of immobility, such as was also found with imipramine, only after pretreatment with naloxone. As shown for morphine, opioids lead to a prolonging of the duration of immobility. This explains why (1R,2R)-3-(2-dimethylaminomethyl-cyclohexyl)-phenol without naloxone pretreatment causes, as the net effect of uptake inhibition and opioid action, only a slight change in the duration of immobility compared with the solvent control.

According to the present findings, (1R,2R)-3-(2-dimethylaminomethyl-cyclohexyl)-phenol surprisingly is the first opiod analgesic to have a relevant and evidently also clinically useful antidepressant action component.

### Example 14

### Parenteral Administration Form

1 g (1R,2R)-3-(2-dimethylaminomethyl-cyclohexyl)-phenol; hydrochloride is dissolved in 1 l of water for injection purposes at room temperature and the solution is then adjusted to isotonic conditions by addition of NaCl.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of treating depression in a mammal, said method comprising administering to said mammal an effective amount of a compound selected from the group consisting of:
3-(2-dimethylaminomethyl-cyclohexyl)-phenol,
(1R,2R)-3-(2-dimethylaminomethyl-cyclohexyl)-phenol,
[2-(3-methoxyphenyl)-cyclohexylmethyl]-dimethylamine,
(1R,2R)42-(3-methoxyphenyl)-cyclohexylmethyl]-dimethylamine,
sulfuric acid mono-[3-(2-dimethylaminomethyl-cyclohexyl)-phenyl]ester,
sulfuric acid mono-(1R,2R)43-(2-dimethylaminomethyl-cyclohexyl)-phenyl]ester,
3-(2-methylaminomethyl-cyclohexyl)-phenol,
(1R,2R)-3-(2-methylaminomethyl-cyclohexyl)-phenol,
3-(2-dimethylaminomethyl-cyclohexyl)-phenol N-oxide,
(1R,2R)-3-(2-dimethylaminomethyl-cyclohexyl)-phenol N-oxide,
6-[3-(2-dimethylaminomethyl-cyclohexyl)-phenoxy]-3,4,5-trihydroxy-tetrahydropyran-2-carboxylic acid,
6-[(1R,2R)-3-(2-dimethylaminomethyl-cyclohexyl)-phenoxy]-3,4,5-trihydroxy-tetrahydropyran-2-carboxylic acid,
4-(2-dimethylaminomethyl-cyclohexyl)-catechol,
(1R,2R)-4-(2-dimethylaminomethyl-cyclohexyl)-catechol,
3-(2-aminomethyl-cyclohexyl)-phenol,
(1R,2R)-3-(2-aminomethyl-cyclohexyl)-phenol,
4-(2-dimethylaminomethyl-cyclohexyl)-benzene-1,2-diol,
(1R,2R)-4-(2-dimethylaminomethyl-cyclohexyl)-benzene-1,2-diol,
C-[2-(3-methoxy-phenyl)-cyclohexyl]-methylamine,
(1R,2R)—C42-(3-methoxy-phenyl)-cyclohexyl]-methylamine,
[2-(3-methoxy-phenyl)-cyclohexylmethyl]-methyl-amine,
(1R,2R)42-(3-methoxy-phenyl)-cyclohexylmethyli-methyl-amine,
[2-(3-methoxy-phenyl)-cyclohexylmethyll-dimethyl-amine N-oxide, and
(1R,2R)-[2-(3-methoxy-phenyl)-cyclohexylmethyl]dimethyl-amine N-oxide
or a salt thereof with a physiologically tolerated acid.

2. A method according to claim 1, wherein said compound is in the form of an R,R stereoisomer.

3. A method according to claim 1, wherein said compound is in the form of an 1R,2R stereoisomer.

4. A method according to claim 1, wherein said compound is in the form of a free base.

5. A method according to claim 1, wherein said compound is in the form of an isolated enantiomer or isolated diastereoisomer.

6. A method according to claim 1, wherein said compound is in the form of a mixture of stereoisomers.

7. A method according to claim 1, wherein said compound is in the form of a racemic mixture.

8. A method according to claim 1, wherein said compound is in the form of a solvate.

9. A metabolite of 3-(2-dimethylaminomethyl-cyclohexyl)-phenol selected from the group consisting of:
sulfuric acid mono-[3-(2-dimethylaminomethyl-cyclohexyl)-phenyl]ester,
sulfuric acid mono-(1R,2R)-{3-(2-dimethylaminomethyl-cyclohexyl)-phenyl]ester,
3-(2-methylaminomethyl-cyclohexyl)-phenol,
(1R,2R)-3-(2-methylaminomethyl-cyclohexyl)-phenol,
3-(2-dimethylaminomethyl-cyclohexyl)-phenol N-oxide,
(1R,2R)-3-(2-dimethylaminomethyl-cyclohexyl)-phenol N-oxide,
6-[3-(2-dimethylaminomethyl-cyclohexyl)-phenoxy]-3,4,5-trihydroxy-tetrahydropyran-2-carboxylic acid,
6-[(1R,2R)-3-(2-dimethylaminomethyl-cyclohexyl)-phenoxy]-3,4,5-trihydroxy-tetrahydropyran-2-carboxylic acid,
4-(2-dimethylaminomethyl-cyclohexyl)-catechol,
(1R,2R)-4-(2-Dimethylaminomethyl-cyclohexyl)-catechol,
3-(2-aminomethyl-cyclohexyl)-phenol,
(1R,2R)-3-(2-aminomethyl-cyclohexyl)-phenol,
4-(2-dimethylaminomethyl-cyclohexyl)-benzene-1,2-diol,
(1R,2R)-4-(2-dimethylaminomethyl-cyclohexyl)-benzene-1,2-diol,
C-[2-(3-methoxy-phenyl)-cyclohexyl]-methylamine,
(1R,2R)—C42-(3-methoxy-phenyl)-cyclohexylFmethylamine,
[2-(3-methoxy-phenyl)-cyclohexylmethyl]-methyl-amine,
(1R,2R)-[2-(3-methoxy-phenyl)-cyclohexylmethyl]-methyl-amine,
[2-(3-methoxy-phenyl)-cyclohexylmethyl]-dimethyl-amine N-oxide and
(1R,2R)-[2-(3-methoxy-phenyl)-cyclohexylmethyl]dimethyl-amine N-oxide
or a salt thereof with a physiologically tolerated acid.

10. The metabolite of claim 9, wherein the metabolite is in the form of an R,R stereoisomer.

11. The metabolite of claim 9, wherein the metabolite is in the form of an 1R,2R stereoisomer.

12. The metabolite of claim 9, wherein the metabolite is in the form of a free base.

13. The metabolite of claim 9, wherein the metabolite is in the form of an isolated enantiomer or isolated diastereoisomer.

14. The metabolite of claim 9, wherein the metabolite is in the form of a mixture of stereoisomers.

15. The metabolite of claim 9, wherein the metabolite is in the form of a racemic mixture.

16. The metabolite of claim 9, wherein the metabolite is in the form of a solvate.

17. A pharmaceutical formulation comprising as an active ingredient at least one metabolite according to claim 9 and a pharmaceutically acceptable auxiliary substance.

18. A pharmaceutical formulation for the treatment of pain comprising as an active ingredient at least one metabolite according to claim 9 and a pharmaceutically acceptable auxiliary substance.

19. A pharmaceutical formulation according to claim 18, wherein said pain is acute pain, visceral pain, chronic pain, neuropathic pain, or cancer pain.

20. A pharmaceutical formulation for the treatment of an increased urge to urinate or urinary incontinence comprising as an active ingredient at least one metabolite according to claim 9 and a pharmaceutically acceptable auxiliary substance.
